(19) United States
(12) Patent Application Publication
(10) Pub. No.: US 2016/0117940 A1
(43) Pub. Date: Apr. 28, 2016

(54) METHOD, SYSTEM, AND APPARATUS FOR TREATING A COMMUNICATION DISORDER

(71) Applicant: LINGRAPHICARE AMERICA INCORPORATED, Princeton, NJ (US)

(72) Inventors: Andrew Gomory, Princeton, NJ (US); Richard Steele, Spokane, WA (US); R. Maxwell Flaherty, Auburndale, FL (US); J. Christopher Flaherty, Auburndale, FL (US)

(21) Appl. No.: 14/427,991

(22) PCT Filed: Aug. 29, 2013
(86) PCT No.: PCT/US13/57178

§ 371 Date: Oct. 7, 2015

Related U.S. Application Data
(60) Provisional application No. 61/700,155, filed on Sep. 12, 2012.

(51) Int. Cl. G09B 5/06; G09B 17/00; G09B 7/00; A61B 5/00; A61B 5/0205; A61B 5/053; A61B 3/028; A61B 5/16; G09B 19/04; A61B 5/11

(52) U.S. Cl. CPC G09B 5/06; G09B 19/04; G09B 17/003; G09B 7/00; A61B 5/11; A61B 5/0205; A61B 5/0533; A61B 3/028; A61B 5/1124; A61B 5/165; A61B 5/167; A61B 5/4803; A61B 5/7275

(57) ABSTRACT

A system, method, and apparatus for treating a communication disorder includes a user input assembly, a central processing unit configured to analyze data entered into the input assembly, and a user output assembly configured to generate a report reflecting the analysis of the data.

### CLAIM OF PRIORITY

This applications is a National phase application of PCT application no. PCT/US13/57178, filed on Aug. 29, 2013. The PCT application PCT/US13/57178 further claims priority to U.S. Provisional Application Ser. No. 61/700,155, filed Sep. 12, 2012, which is incorporated herein by reference, in its entirety.

### FIELD OF TECHNOLOGY

The present invention relates generally to speech and language systems and methods, and more particularly to systems and methods for treating or assisting a patient with a communication disease or disorder.

### BACKGROUND

Impairment of language is common among patients who have suffered from a traumatic brain injury such as a head injury or stroke. For example, aphasia is an impairment of language ability where a patient may have an impairment ranging from a difficulty remembering words to a complete inability to speak, read, or write. Typical behaviors include inability to comprehend language, inability to pronounce syllables or words, inability to speak spontaneously, inability to form words, inability to name objects, poor enunciation, excessive creation and use of personal neologisms, inability to repeat a phrase, and persistent repetition of phrases.

Apraxia of speech, often accompanies aphasia after a stroke. This type of speech impairment affects the ability to plan and coordinate the movements necessary for speech. Typically, a patient suffering from a language impairment is treated by a speech therapist, for example a Speech and Language Pathologist (SLP), however language impairments can be very difficult to treat, and specifically, patients with moderate to severe aphasia and/or apraxia often require years of therapy with slow progress and variable outcomes. Therefore devices, systems and methods that provide both language augmentation to help users function better in their daily activities as well as cuing and practice that are particularly suited for aphasia and apraxia of speech are desirable.

### SUMMARY

According to an aspect of the present invention, a system for a patient includes: a user input assembly constructed and arranged to allow a user to enter input data; an input data analyzer constructed and arranged to receive the input data, analyze the input data and produce results based on the input data; a user output assembly constructed and arranged to receive the results from the input data analyzer and display the results; and a data library constructed and arranged to receive the results from the input data analyzer and store the results.

The system can be constructed and arranged to treat a patient having a communication disorder comprising a disorder selected from the group consisting of: aphasia; apraxia of speech; dysarthria; dysphagia; and combinations of these. Aphasia can be selected from the group consisting of: global aphasia; isolation aphasia; Broca's aphasia; Wernicke's aphasia; transcortical motor aphasia; transcortical sensory aphasia; conduction aphasia; anomic aphasia; primary progressive aphasia; and combinations of these.

The system can be further constructed and arranged to treat a condition selected from the group consisting of: conditions of motor involvement such as right hemiplegia; sensory involvement such as right hemianopsia and altered acoustic processing; cognitive involvement such as memory impairments, judgment impairments and initiation impairments; and combinations of these.

The communication disorder can comprise a disorder caused by at least one of: a stroke; a trauma to the brain or a congenital disorder; a medical accident or side effect thereof; a traumatic brain injury; a penetrating head wound; a closed head injury; a tumor; a medical procedure adverse event; an adverse effect of medication.

The system can be constructed and arranged to provide a benefit to the patient such as a therapeutic benefit; an orthotic benefit; a prosthetic benefit; and combinations of these.

The user input assembly can comprise an assembly selected from the group consisting of: microphone; mouse; keyboard; touchscreen; camera; eye tracking device; joystick; trackpad; sip and puff device; gesture tracking device; brain machine interface; any computer input device; and combinations of these.

The user can include a user selected from the group consisting of: a speech language pathologist; the patient; a second patient; a representative of the system manufacturer; a family member of the patient; a support group member; a clinician; a nurse; a caregiver; a healthcare statistician; a hospital; a healthcare insurance provider; a healthcare billing service; and combinations of these. A user can include multiple users, for example a patient and a speech and language pathologist. Another example includes a first patient and a second patient.

The input data can comprise at least recorded speech, for example where the speech represents at least one of: a sentence; a word; a partial word; a phonetic sound such as a diphone, a triphone or a blend; a phoneme; or a syllable. The input data can also include data selected from the group consisting of: written data; patient movement data such as lip or tongue movement data; patient physiologic data; patient psychological data; patient historic data; and combinations of these.

The input data can comprise at least patient written data, for example written data comprising data generated by the patient selected from the group consisting of: a picture; text; written words; icons; symbols; and combinations of these.

The input data can comprise at least patient movement data, for example data recorded from video camera; data recorded from keypad or keyboard entry; data recorded from mouse movement or clicking; eye movement data; and combinations of these.

The input data can comprise at least patient physiologic information, for example monitored galvanic skin response; respiration; EKG; visual information such as left field cut and right field cut; macular degeneration; lack of visual acuity; limb apraxia; limb paralysis; and combinations of these.

The input data can comprise at least patient psychological information, for example Meyer-Briggs personality structure data; Enneagram type data; diagnostic and other data related to disorders such as cognitive disorders and memory disorders; data representing instances of depression; and combinations of these.

The input data can comprise at least patient historic data, for example patient previous surgery or illness data; family medical history; and combinations of these.

The input data can comprise at least external data, for example medical reference data such as medical statistics from one or more similar patient populations; medical literature relevant to the patient disorder; user billing data; local or world news; any data available via the internet; and combinations of these.

The user output assembly can comprise an output assembly selected from the group consisting of: a visual display; a touchscreen; a speaker; a tactile transducer; a printer for generating a paper printout; and combinations of these.

The data analyzer analysis can comprise a manual analysis step performed by an operator. The manual analysis step can be performed at a location remote from the patient. The manual analysis step can be performed at approximately the same time that the input data is entered; within one hour that the input data is entered; at least four hours after the input data is entered; or at least twenty-four hours after data is entered. The data analyzer analysis can comprise an at least partially automated analysis, for example where the data analyzer analysis comprises at least one manual analysis step and at least one automated analysis step. Alternatively, the data analyzer analysis can be a fully automated analysis. The data analyzer analysis can comprise a quantitative analysis.

The results produced by the data analyzer can comprise a status of the patient's disease state. The status of the patient's disease can be assessed via at least one of: involvement indications and profiles from WHO taxonomy of disease such as impairment, activity limitation, and participation restriction; WAB; BDAE; PICA; BNT; PNT; ASHA-FACS; ASHA-QOCL; and other standardized or non-standardized assessments of aphasia and other language disorders, language impairment, functional communication, satisfaction, and quality of life.

The results can comprise an assessment of improvement of the patient's disease state. The improvement of the patient's disease can be assessed via at least one of: improvements as revealed by statistical analyses of assessments from WHO taxonomy of disease such as impairment, activity limitation, and participation restriction; WAB; BDAE; PICA; BNT; PNT; ASHA-FACS; ASHA-QOCL; other standardized assessments of aphasia; and data from non-standardized assessment instruments such as the PALPA.

The results can comprise an assessment of a patient therapy. In some embodiments, the system further comprises a therapy application constructed and arranged to provide the patient therapy.

The results can comprise an assessment of a first patient therapy and a second patient therapy. In some embodiments, the system further comprises a therapy application constructed and arranged to provide at least the first patient therapy.

The results can comprise an assessment of a patient parameter selected from the group consisting of: functional communication level; speech impairment level; quality of life; impairment, activity limitation; participation restriction; and combinations of these.

The results can comprise a patient prognosis, for example a prognosis of at least one of future disease state status; disease state progression; prognostic indications from scientific analyses of data gathered using WHO taxonomy of disease such as impairment, activity limitation, and participation restriction; WAB; BDAE; PICA; BNT; PNT; ASHA-FACS; ASHA-QOCL; other standardized or non-standardized assessments of aphasia. In some embodiments, the system can further comprise a therapy application, where the prognosis comprises an estimation of expected improvement after using the therapy application. In some embodiments, the system can comprise a first therapy application and a second therapy application, where the prognosis compares the expected improvement to be achieved with the first therapy application with the expected improvement to be achieved with the second therapy application.

The results can comprise a report similar to a standard communication disorder assessment test report. For example a report generated from a standardized assessment such as a scientific analyses of data gathered using WHO taxonomy of disease such as impairment, activity limitation, and participation restriction; WAB; BDAE; PICA; BNT; PNT; ASHA-FACS; ASHA-QOCL; and other standardized or non-standardized assessments of aphasia. The results can comprise a report which can be correlated to the standard communication disorder assessment test report described in this paragraph.

The results can comprise a playback of recorded data, for example: video recordings, audio recordings, movement data such as keystroke entry data; and combinations of these. The recorded data playback can be manipulated by at least one of: a fast forward manipulation; a rewind manipulation; a play manipulation; a stepped-playback manipulation; or a pause manipulation.

The results can comprise a summary of keystrokes, for example a summary of keystrokes made by at least the patient user of the system or at least a non-patient user of the system.

The results comprise at least a numerical score; a qualitative score; a representation of a pattern of error; an analysis of a patient's speech; an analysis of patient written data; an analysis of patient answer choices, for example patient answer choices to a therapy program; an analysis of at least one of keystrokes; mouse clicks; body movement such as lip, tongue or other facial movement; touch screen input; and combinations of these. In some embodiments, the system can further comprise a therapy application, where the results comprise an analysis of at least one of a time duration of the therapy application; or elapsed time between segments of the therapy application.

The system can further comprise a therapy application. The therapy application can comprise multiple user selectable levels, for example at least a first level; a second level more difficult than the first level; and a third level more difficult than the second level. The therapy application can comprise multiple user selectable therapy sub-applications, for example a first therapy sub-application that comprises different content than a second therapy sub-application. Examples of content include: motion picture content; trivia content; sports information content; historic information content; and combinations of these. The first therapy sub-application can comprise different functionality than the second therapy sub-application. Examples of functions include: mail function such as an email function; phone function such as internet phone function; news retrieval function; word processing function; accounting program function such as bill paying function; video or other game playing function; and combinations of these. The first therapy sub-application comprises different patient provided information than the second therapy sub-application, for example a difference in at least one of: icons displayed; pictures displayed; text displayed; audio provided; or moving video. The different patient information can be based on an adaptive signal processing of ongoing user performance.

Examples of therapy applications include: a linguistic therapy application; a syntactic therapy application; an auditory comprehension therapy application; a reading therapy application; a speech production therapy application; a cognitive therapy application; a cognitive reasoning therapy application; a memory therapy application; a music therapy application; a video therapy application; a lexical therapy application exercising items such as word length, number of syllables, segmental challenge levels, phonotactic challenge levels, word frequency data, age of acquisition data, iconic transparency, and iconic translucency; and combinations of these. The type of therapy application can be selected based on a patient diagnostic evaluation and/or based on a communication disorder diagnosed for the patient.

The therapy application can comprise a first application and a second application, where the system is constructed and arranged to adapt the second therapy application based on the first therapy application. For example, the second therapy application can be constructed and arranged to adapt based on the results. The second therapy application can be manually adapted based on the assessment of a speech language pathologist. The second therapy application can be manually adapted based on a patient self-assessment. The second therapy application can be automatically adapted by the system, for example where the system automatic adaptation of the second therapy application is based on at least one of: quantitative analysis of results produced during first therapy application; qualitative analysis of results produced during first therapy application; or indicated clinical pathways based on correlations of application sequencing and improvement types, magnitudes, and change rates.

The therapy application can comprise a series of therapy applications performed prior to the performance of a single therapy application, where the single therapy application can be adapted based on the multiple therapy applications. The single therapy application can be adapted based on at least one of: the cumulative results of the multiple therapy applications; the averaged results of the multiple therapy applications; the standard deviation of the results of the multiple therapy applications; or a trending analysis of the results of the multiple therapy applications.

The system can be constructed and arranged to provide the data library to at least one of: the patient; a speech language pathologist; a caregiver; a support group representative; a clinician; a physician; the system manufacturer; a hospital; a health information system; a system component; or a system assembly. The data library can be at least one of: downloadable; transferable; printable; or recoverable. The data library can comprise a permission-based access data library. The data library can comprise a speech-to-text data set of information, for example a data set customized for a patient diagnosed condition, a data set customized for a patient pre-existing accent, and/or a data set including a limited choice of pre-identified words. The data library can be constructed and arranged to store data selected from the group consisting of input data; results; patient historic data; external data such as word libraries and medical reference data; and combinations of these.

The system can further comprise a configuration algorithm. The configuration algorithm can be constructed and arranged to configure the system with user-specific modifications such as patient-specific modifications. The configuration algorithm can be constructed and arranged to allow an operator to set a difficulty level for the system. The configuration algorithm can modify one or more system parameters automatically, for example a modification based on the results. The configuration algorithm can modify one or more system parameters manually, for example the patient or a speech language pathologist can modify one or more system parameters.

The system can further comprise a threshold algorithm. The threshold algorithm can be constructed and arranged to cause the system to enter an alarm state if one or more parameters fall outside a threshold. In some embodiments, the system further comprises a therapy application, where the threshold algorithm can be constructed and arranged to cause the system to modify the therapy application if one or more parameters fall outside a threshold. The threshold algorithm can be constructed and arranged to compare a parameter to a threshold wherein the parameter is selected from the group consisting of: lexical parameters such as word length, number of syllables, segmental challenge, phonotactic challenge, abstractness, and age of acquisition; syntactic parameters such as mean length of utterance, phrase structure complexity, and ambiguity metrics; pragmatic parameters such as contextual interpretation support, and salience for particular patient; number or percentage of incorrect answers; number or percentage of correct answers; time taken to perform a task; user input extraneous to completing a task; period of inactivity; time between user input events; hesitation pattern analysis; and combinations of these.

The system can further comprise a self-diagnostic assembly. The self-diagnostic assembly can comprise at least a software algorithm. The self-diagnostic assembly can comprise at least a hardware assembly. The self-diagnostic assembly is constructed and arranged to detect at least one of: power failure; inadequate signal level such as inadequate signal level recorded at the user input assembly; interruption in data gathering; factors affecting human performance such as distraction, fatigue, and aggravation; interruption in data transmission; unexpected system failure; or unexpected termination of a user task.

The system can further comprise a report generator. The report generated can comprise a representation of the results, for example a graphical representation; a representation of percentages; a representation of comparisons; and combinations of these. The report can comprise a comparison of results, for example comparison of results from the same patient; comparison of results from the patient and a different patient; comparison of results from the patient to a summary of multiple patient data; and combinations of these.

The system can further comprise a non-therapy application. Examples of non-therapy applications include: a picture-based electronic communication tool; an audio-based electronic communication tool; a game such as a video game; a news information reader; a telephone internet program; a location-based application using GPS information to provide stimuli for various purposes such as information review, provision of utterance feedback, stimuli to cue functional speech and the like; and combinations of these. The system can be constructed and arranged to use the input data to control the non-therapy application.

The system can further comprise a patient health monitor. The patient health monitor can be constructed and arranged to detect one or more patient physiologic parameters and/or speech or motor functions indicative of an adverse event based on the input data, for example where the adverse event comprises a stroke.

The system can further comprise a one-click emergency button constructed and arranged to allow a user to contact an emergency handling provider.

The system can further comprise a mute-detection algorithm constructed and arranged to detect an inadvertent mute condition of the at least one microphone, for example where the user input assembly can comprise the at least one microphone. The mute-detection algorithm can be further constructed and arranged to contact an emergency handling provider.

The system can further comprise an automated speech language pathologist function, for example where the function is selected from the group consisting of: patient assessment; disease diagnosis; treatment plan creation; delivery of treatment; reporting of treatment delivered; data gathered on patient performance; ongoing reassessment and change to treatment plan; outcome and/or progress prognosis; outcome and/or progress measurement; and combination of these.

The system can further comprise a remote control function. The remote control function can be constructed and arranged to allow a representative of the system manufacturer to perform a function selected from the group consisting of: log in to a system component; control a system component; troubleshoot a system component; train a patient; and combinations of these.

The system can further comprise a login function constructed and arranged to allow a user to access the system by entering a username. The login function can comprise a password algorithm. The system can be constructed and arranged to allow multiple levels of authorization based on the username.

The system can further comprise an external input assembly constructed and arranged to receive external data from an external source. The input data analyzer can be further constructed and arranged to receive the external data, analyze the external data and produce results based on the external data. The external input assembly can be constructed and arranged to receive the external data via at least one of: a wire; the internet; or a wireless connection such as Bluetooth or cellular service connection.

The system can further comprise a timeout function constructed and arranged to detect a pre-determined level of user inactivity, for example, the system can be constructed and arranged to modify a system parameter if a level of user inactivity is detected by the timeout function. Additionally, the system can constructed and arranged to contact an emergency handling provider.

The system can further comprise other patient data where the data analyzer can be further constructed and arranged to receive the other patient data and analyze the other patient data. The input data analyzer can be further constructed and arranged to produce the results based on the other patient data. In some embodiments, the system further comprises a therapy application and/or a non-therapy application where the application can be based on the other patient data.

The system can comprise a family mode. In some embodiments, the system further comprises a therapy application where the family mode can be constructed and arranged to allow a patient family member to participate in the therapy application.

The system can comprise a multiple patient mode. In some embodiments, the system further comprises a therapy application wherein the multiple patient mode can be constructed and arranged to allow multiple patients to participate in the therapy application.

The system can comprise a multiple caregiver mode. The multiple caregiver mode is constructed and arranged to support multiple users selected from the group consisting of: a therapist such as a speech language therapist; a physical therapist; a psychologist; a general practitioner; a neurologist; and combinations of these. The multiple caregiver mode can be constructed and arranged to allow multiple caregivers to access the system at least one of simultaneously or sequentially.

The system can be constructed and arranged to allow transfer of control from a first user to a second user. For example, where the first user is a patient caregiver and the second user is a patient caregiver, the system can be constructed and arranged to allow transfer of control from the first user to the second user. A third user can be included, for example where a third user is a patient and the first user and second user are caregivers of the third user.

The system can further comprise a billing algorithm. The billing algorithm can comprise a pay per user algorithm. The billing algorithm can comprise a pay per time period algorithm. The billing algorithm can comprise a discount based on at least one of user feedback; extended personal information provided by a user; or user assessments.

The system can further comprise an email function.

The system can further comprise a speech to text algorithm, for example an algorithm which is biased by at least one of: accent; disability; slur; stutter; stammer; performance effects of communication disorders such as apraxia of speech, dysarthria, and dysphagia; or organic damage to articulators.

The system can further comprise a user interface. The user interface can be constructed and arranged to adapt during use. In some embodiments, the system further comprises a diagnostic function where the user interface adapts based on the diagnostic function. The user interface can adapt based on patient performance during use. The user interface can be manually modified by a user.

According to another aspect of the present invention, a method for a patient includes using the system described above to treat a communication disorder of the patient. The method can be performed in any specified language. Additionally or alternatively, the method can be performed in English.

According to another aspect of the present invention, a method for a patient includes using the system described above to provide a therapeutic benefit to the patient.

According to another aspect of the present invention, a method for a patient includes using the system described above to provide an orthotic benefit to the patient.

According to another aspect of the present invention, a method for a patient includes using the system described above to provide a prosthetic benefit to the patient.

According to another aspect of the present invention, a method for a patient includes analyzing patient data and selecting a therapy based on the analysis. The input data can comprise at least recorded speech, for example where the speech represents at least one of: a sentence; a word; a partial word; a phonetic sound such as a diphone, a triphone or a blend; a phoneme; or a syllable. The input data can also include data selected from the group consisting of: written data; patient movement data such as lip or tongue movement data; patient physiologic data; patient psychological data; patient historic data; and combinations of these.

The patient data can comprise at least patient written data, for example written data comprising data generated by the patient selected from the group consisting of: pictures; text; written words; icons; symbols; and combinations of these.

The patient data can comprise at least patient movement data, for example data recorded from video camera; data recorded from keypad or keyboard entry; data recorded from mouse movement or clicking; eye movement data; and combinations of these.

The patient data can comprise at least patient physiologic information, for example monitored galvanic skin response; respiration; EKG; visual information such as left field cut and right field cut; macular degeneration; lack of visual acuity; limb apraxia; limb paralysis; and combinations of these.

The patient data can comprise at least patient psychological information, for example Meyer-Briggs personality structure data; Enneagram type data; diagnostic and other data related to disorders such as cognitive disorders and memory disorders; data representing instances of depression; and combinations of these.

The patient data can comprise at least patient historic data, for example patient previous surgery or illness data; family medical history; and combinations of these.

The patient data can comprise at least external data, for example medical reference data such as medical statistics from one or more similar patient populations; medical literature relevant to the patient disorder; user billing data; local or world news; any data available via the internet; and combinations of these.

Selecting the therapy can be based on results selected from the group consisting of: diagnostic procedure results; cumulative results of multiple therapy applications; averaged results of multiple therapy applications; standard deviation of results of multiple therapy applications; a trending analysis of results of multiple therapy applications; and combinations of these.

Selecting the therapy can comprise adapting a second therapy application based on a first therapy application.

The therapy can comprise a therapy application and the therapy selection can comprise selecting a therapy application level, for example where the level comprises at least a first level; a second level more difficult than the first level; and a third level more difficult than the second level. The therapy can comprise a therapy application and the therapy selection can comprise a selection of a therapy sub-application, for example where a first therapy sub-application can comprise different content than a second therapy sub-application. Examples of content include: motion picture content; trivia content; sports information content; historic information content; and combinations of these. The first therapy sub-application can comprise a different functionality than the second therapy sub-application. Examples of functions include: a function selected from the group consisting of: phone function such as internet phone function; news retrieval function; word processing function; accounting program function; and combinations of these. The first therapy sub-application can comprise different patient provided information than the second therapy sub-application, for example icons displayed; pictures displayed; text displayed; audio provided; or moving video provided.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a schematic of a system for treating a communication disease or disorder, consistent with the present inventive concepts.

FIG. 2 illustrates a method for treating a communication disease or disorder, consistent with the present inventive concepts.

FIG. 3 illustrates a method for diagnosing a patient for a communication disease or disorder, consistent with the present inventive concepts.

FIG. 4 illustrates a schematic of a system for treating a communication disease or disorder including multiple users, consistent with the present inventive concepts.

FIG. 5 illustrates a method for determining the appropriate level of therapy for a patient with a communication disease or disorder, consistent with the present inventive concepts.

FIG. 6 illustrates a self-diagnostic algorithm, consistent with the present inventive concepts.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

The systems and methods disclosed herein can be used to treat various communication diseases or disorders (hereinafter “disorders”), as well as conditions commonly associated with those disorders. Communication disorders can be caused by a traumatic brain injury such as a head injury or stroke; a congenital disorder; a medical accident or side effect thereof; a penetrating head wound; a closed head injury; a tumor; a medical procedure adverse event; an adverse effect of medication; and combinations of these. Examples of communication disorders include aphasia; apraxia of speech; dysarthria; dysphagia; and combinations of these. Examples of types of aphasia include: global aphasia; isolation aphasia; Broca's aphasia; Wernicke's aphasia; transcortical motor aphasia; transcortical sensory aphasia; conduction aphasia; anomic aphasia; and primary progressive aphasia. Additionally, the systems and methods disclosed herein can be used to treat conditions commonly associated with the above listed communication disorders such as conditions of motor involvement such as right hemiplegia; sensory involvement such as right hemianopsia and altered acoustic processing; cognitive involvement such as memory impairments, judgment impairments and initiation impairments; and combinations of these. In addition to or as an alternative to treating a communication disorder, the systems and methods disclosed herein can serve as a communication tool. The systems and methods of the present inventive concepts can provide numerous benefits to the patient, such as a benefit selected from the group consisting of: a therapeutic benefit; an orthotic benefit; a prosthetic benefit; and combinations of these. The systems and methods of the present inventive concepts can be provided in any language in addition to or alternative to English.

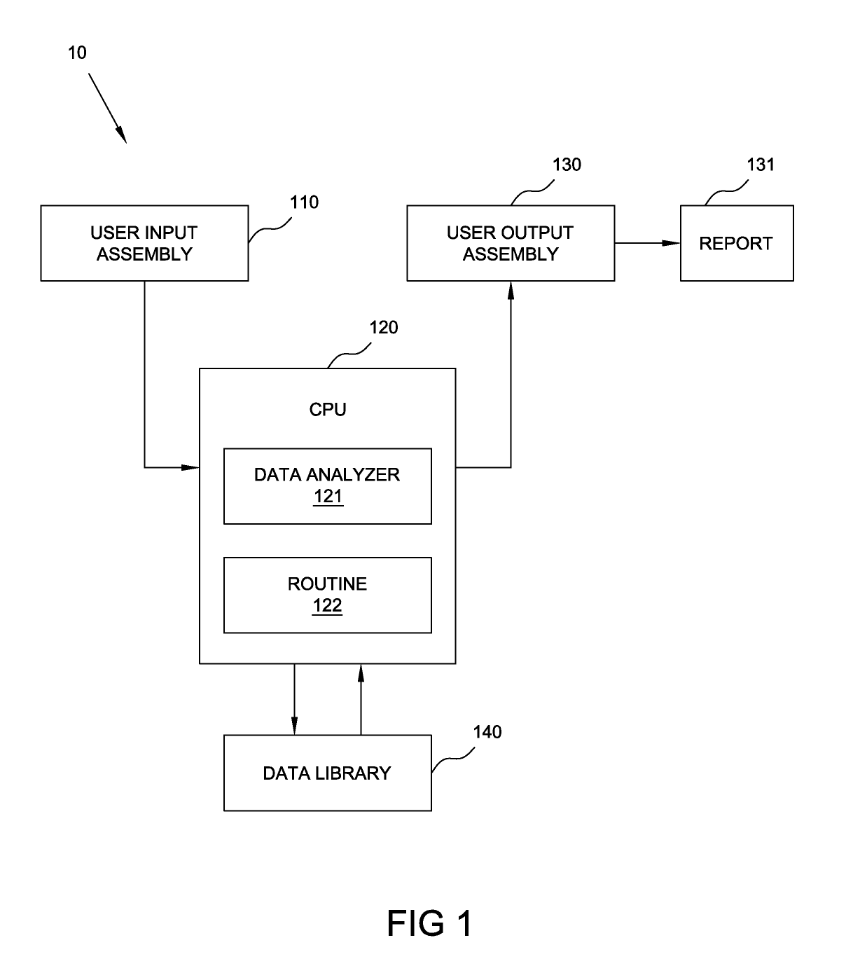

FIG. 1 illustrates a system for treating a communication disorder, consistent with the present inventive concepts. System 10 includes user input assembly 110 configured to allow a user to enter input data. System 10 further includes central processing unit 120, including data analyzer 121, configured to receive and analyze the input data and produce one or more results (hereinafter “results”) based on the data. System 10 further includes user output assembly 130 configured to receive the results from data analyzer 121 and display the results, for example via a report such as report 131. Additionally, system 10 includes data library 140 configured to receive the results from data analyzer 121 and store results, such as for further analysis, comparison with other collected data, or for another future use.

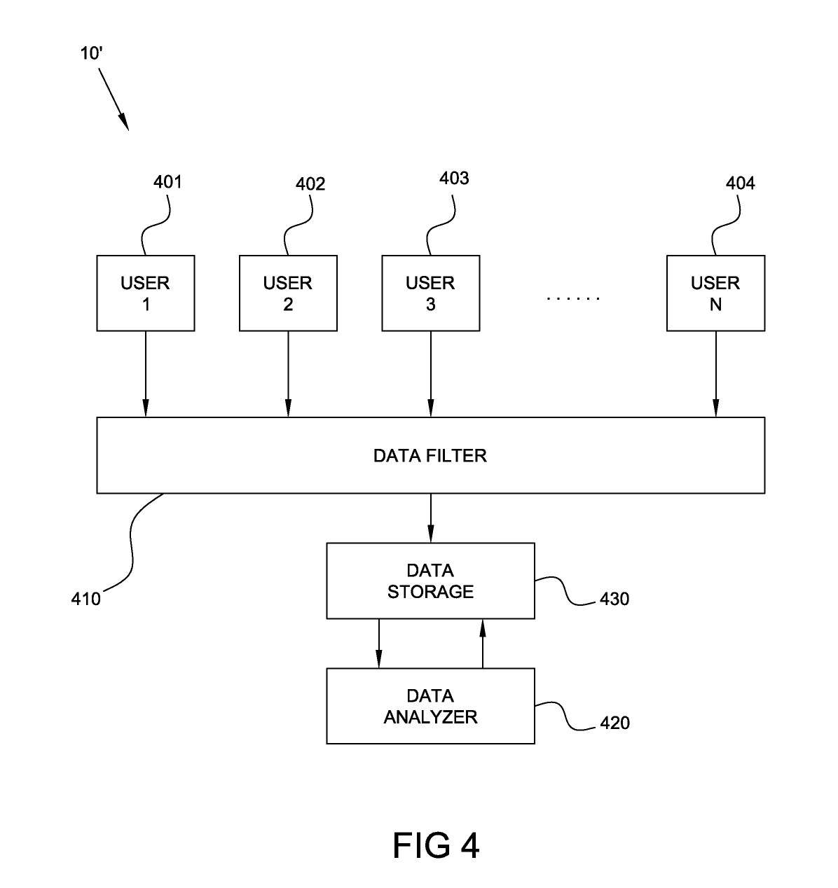

System 10 can be utilized by a single user or multiple users, for example as is described in the embodiment described in FIG. 4. A user can include a speech therapist such as a Speech and Language Pathologist (SLP); a patient; a second patient; a representative of the system manufacturer; a family member of the patient; a support group member; a clinician; a nurse; a caregiver; a healthcare statistician; a hospital; a healthcare insurance provider; a healthcare billing service; and combinations of these.

Input assembly 110 can include: microphone; mouse; keyboard; touchscreen; camera; eye tracking device; joystick; trackpad; sip and puff device; gesture tracking device; brain machine interface; a computer input device; and combinations of these. Data entered into input assembly 110 can include recorded speech, such as speech representing at least one of: a sentence; a word; a partial word; a phonetic sound such as a diphone, a triphone or a blend; a phoneme; or a syllable. Numerous forms of patient and other data can be entered into input assembly 110, such as data selected from the group consisting of: written data; patient movement data such as lip or tongue movement data; patient physiologic data; patient psychological data; patient historic data; and combinations of these. Examples of written data include written data generated by the patient, such as data selected from the group consisting of: a picture; text; written words; icons; symbols; and combinations of these. Examples of patient movement data include: data recorded from a video camera; data recorded from a keypad or keyboard entry; data recorded from mouse movement or clicking; eye movement data; and combinations of these. Examples of patient physiologic data include: Meyer-Briggs personality structure data; Enneagram type data; diagnostic and other data related to disorders such as cognitive disorders and memory disorders; data representing instances of depression; and combinations of these. Examples of patient historic data include: patient previous surgery or illness data; family medical history; and combinations of these. External data can be entered into input assembly 110. Typical external data includes but is not limited to: medical reference data such as medical statistics from one or more similar patient populations; medical literature relevant to the patient disorder; and combinations of these.

Central processing unit 120 is constructed and arranged to perform routine 122 where routine 122 can include a therapy application. The therapy application can be displayed to the user via a user interface enabling the user to perform the therapy application, such as when input device 110 comprises a user interface (e.g. both user input and output components). The therapy application can be used by a patient to improve a communication disorder and/or a condition associated with a communication disorder. Examples of types of therapy applications include: a linguistic therapy application; a syntactic therapy application; an auditory comprehension therapy application; a reading therapy application; a speech production therapy application; a cognitive therapy application; a cognitive reasoning therapy application; a memory therapy application; a music therapy application; a video therapy application; a lexical therapy application exercising items such as word length, number of syllables, segmental challenge levels, phonotactic challenge levels, word frequency data, age of acquisition data, iconic transparency, and iconic translucency; and combinations of these.

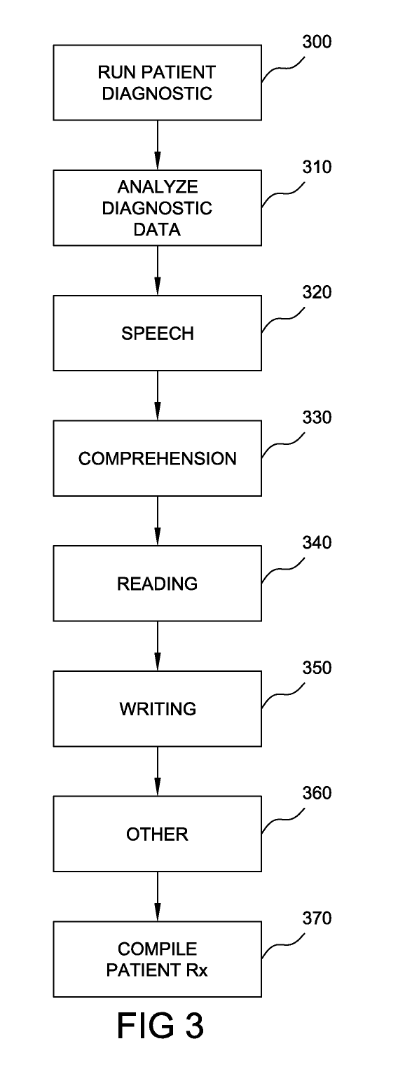

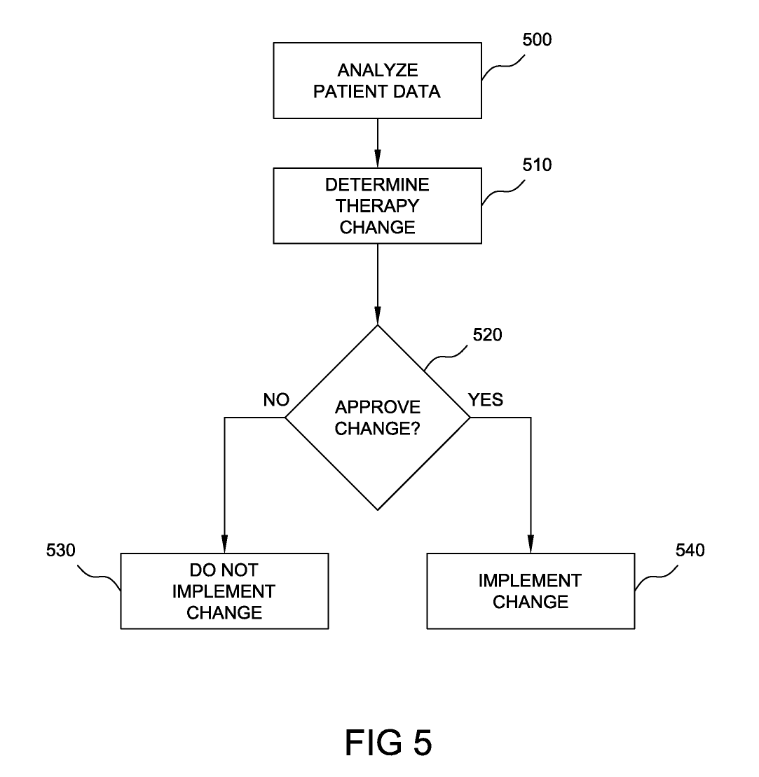

The type of therapy application can be chosen based upon a patient diagnostic evaluation, described in detail in FIG. 3 herebelow. The type of therapy application can be chosen based on the type of communication disorder the patient is diagnosed with. In some embodiments, the therapy application can include a first application and a second application where the second application is adapted based on the results of the first application, the details of these embodiments described in detail in FIG. 5 herebelow.

The therapy application and/or the user interface can be customizable by a user. For example, the therapy application can include multiple user selectable levels, such as “easy”, “medium” and “hard” levels. The therapy application can include multiple user selectable therapy sub-applications, for example where a sub-application includes content such as motion picture content; trivia content; sports information content; historic information content; and combinations of these. Additionally or alternatively, the therapy application can include multiple user selectable therapy sub-applications, where each sub-application includes a different functionality. Examples of applicable functions include but are not limited to: mail function such as an email function; phone function such as internet phone function; news retrieval function; word processing function; accounting program function such as a bill paying function; video or other game playing function; and combinations of these. Additionally or alternatively, the therapy application can include multiple user selectable therapy sub-applications, where each sub-application provides different information to the patient such as icons displayed; pictures displayed; text displayed; audio provided; moving video provided; and combinations of these. In one example, the user (e.g. a patient) can customize the therapy application by selecting the content and functionality of the application and/or interface based on personal preference and/or area of improvement needed. For example, if the patient is at a beginner level and is a sports fan, the patient can select an “easy” level therapy application with content such as icons, pictures, audio, text and/or moving video that is sports-based.

The customization and/or any modification of the user interface and/or therapy application can be manual or automatic based upon one or more parameters or upon data collected through the performance of one or more procedures. For example, the interface and/or application can adapt based upon a diagnostic procedure and/or a user's performance of the therapy application or other therapy application, details of which are described in FIGS. 3 and 5 herebelow.

Central processing unit 120, including data analyzer 121, is configured to perform an analysis of data, such as any or all of the data described in the paragraphs above. In some embodiments, data analyzer 121 includes a manual analysis step that can be performed at a location remote from the patient. The data analysis step can be performed in real time, or after time has elapsed since data was entered into input assembly 110, such as a time within one hour from the time the data was entered; more than four hours after the data was entered; or more than twenty-four hours after the data was entered. In some embodiments, data analyzer 121 includes a fully automated, or at least a partially automated analysis of the data. For example, the analysis can include at least one manual analysis step and at least one automated analysis step. The analysis can include a quantitative and/or a qualitative analysis of the data. Central processing unit 120 can comprise one or more discreet components, such as one or more components at the same locations or at different locations. Central processing unit 120 can include a centralized processing portion, such as available via wired or wireless communication and located on a web server or at the manufacturer. Central processing unit 120 can comprise at least a portion that is included in a device configured for patient ambulation, such as a hand-held electronics device, a cell phone, a personal data assistant, or the like.

Output assembly 130 is configured to produce results. In one embodiment, output assembly 130 can include a report generator where the results can be included in report 131. Output assembly 130 can include a visual display; a touchscreen; a speaker; a tactile transducer; a printer for generating a paper printout; and combinations of these. Report 131 can be similar and/or correlate to a standard communication disorder assessment test report. In some embodiments, assessments include one or more of the following: scientific analyses of data gathered using WHO taxonomy of disease such as impairment, activity limitation, and participation restriction; WAB; BDAE; PICA; BNT; PNT; ASHA-FACS; ASHA-QOCL; and other standardized assessments of aphasia; and data from non-standardized assessment instruments such as the PALPA. Report 131 can display results in a variety of ways, for example via a graphical representation; a representation of percentages; a representation of comparisons; and combinations of these. Examples of comparisons include: comparison of results from the same patient; comparison of results from the patient and a different patient; comparison of results from the patient to a summary of multiple patient data; and combinations of these.

The results can include a status of the patient's disease state, including any improvements thereof, where the patient's disease state is assessed via at least one of: involvement indications and profiles from WHO taxonomy of disease such as impairment, activity limitation, and participation restriction; WAB; BDAE; PICA; BNT; PNT; ASHA-FACS; ASHA-QOCL; and other standardized assessments of aphasia and other language disorders, language impairment, functional communication, satisfaction, and quality of life.

The results can include an assessment of the patient's therapy, for example one or more of the therapy applications of system 10 or another therapy used to treat the patient. The results can include a comparison of two or more therapies being provided to the patient, for example where at least one of the two or more therapies includes a therapy application of system 10. Examples of other therapies include: a session with an SLP or other therapist, a psychological review, a physical therapy session, a group therapy session, and the like. The results can include an assessment of a patient parameter. Examples of patient parameters include: functional communication level; speech impairment level; quality of life; impairment, activity limitation; participation restriction; and combinations of these.

The results can include a patient prognosis. Examples of a patient prognosis include but are not limited to: future disease state status; disease state progression; prognostic indications from scientific analyses of data gathered using WHO taxonomy of disease such as impairment, activity limitation, and participation restriction; WAB; BDAE; PICA; BNT; PNT; ASHA-FACS; ASHA-QOCL; other standardized assessments of aphasia; data from non-standardized assessment instruments such as the PALPA; and combinations of these. The prognosis can include an estimation of expected improvement after using a therapy application, such as the estimated of expected improvement based on duration of therapy or the maximum expected improvement for that patient with therapy. The prognosis can include a comparison of the expected improvement to be achieved with a first therapy application versus the expected improvement to be achieved with a second therapy application, such as to choose a course of therapy or eliminate one or more therapy programs.

System 10 can be constructed and arranged to provide a playback of recorded data or other results. Examples of recorded data include: video recordings; audio recordings; movement data such as keystroke entry data; and combinations of these. Examples of playback options include: a fast forward manipulation; a rewind manipulation; a play manipulation; a stepped-playback manipulation; a pause manipulation; and combinations of these.

The results can include a summary of keystrokes made by a user, such a patient or a non-patient user. For example, the summary of keystrokes can include a summary of the keystrokes made by the patient during a therapy application.

The results can include an analysis of written data; speech; keystrokes; mouse clicks; body movement such as lip, tongue or other facial movement; touch screen input; and combinations of these. The results can include an analysis of user answer choices, for example where a therapy application includes one or more questions requiring an answer from the user. The results can include an analysis of the number of correct answers made by the patient in response to questions posed by system 10. The results can include an analysis of the total time it takes for a user to complete a therapy application or any portions or segments thereof. Also, the results can include an analysis of any elapsed time between portions or segments of a therapy application, for example if a therapy application of system 10 includes twenty multiple choice questions, the results can include an analysis of elapsed time between any or all patient responses to those questions.

The results can represent a pattern of error as well as whether the pattern of error is due to a user of system 10, such as a patient using system 10, versus another error such as a faulty component of system 10. For example, a user typing on a keyboard intends to type the word “school”, however repeatedly types “scgool”. Data analyzer 121 can detect this pattern of error and additionally detect whether the error is due to the user's mistyping the word, since the “g” key is directly to the left of the “h” key on a traditional keyboard, or if the error is due to an error in the commands, i.e. the “g” key and the “h” key are not transmitting the proper commands. One example of how the algorithm can determine if the error is due to the system or the user is by searching for other words that include the letter “h” and/or the letter “g” and compare.

Based on the above mentioned analyses and summaries, the results can score the patient's performance of a therapy application, for example a numerical score and/or a qualitative score.

The results and/or report 131 can be reviewed by a therapist, for example an SLP who performs a function such as: patient assessment; disease diagnosis; treatment plan creation; delivery of treatment; reporting of treatment delivered; data gathered on patient performance; ongoing reassessment and change to treatment plan; outcome and/or progress prognosis; outcome and/or progress measurement; and combinations of these. In some embodiments, system 10 further includes an automated speech language pathologist function configured to perform the above listed functions, either alone or in combination with the SLP.

System 10 can include data library 140 configured to store information, including input data; results; patient historic data; external data; and combinations of these. Any or all of the data can be downloadable, transferable, printable or otherwise recovered and/or used at a future time.

Data library 140 can store a speech-to-text data set of information, where the data set can be created using an algorithm that is customized to the patient. In some embodiments, the algorithm is biased or otherwise customized for a patient diagnosed condition; a patient pre-existing accent; a limited choice of pre-identified words; and/or combinations of these. For example, if the user has an accent and is performing a therapy application where speech is required to complete the application, data library 140 can recognize or otherwise adjust for the user's particular accent, such as via a data set that has been customized for the accent.

Data library 140 can be accessed by at least one of: the patient; an SLP; a caregiver; a support group representative; a clinician; a physician; system 10 manufacturer; a hospital; a health information system; a system 10 component; or a system 10 assembly. Access to data library 140 can be permission-based, such as requiring a username and/or a password.

System 10 can optionally include a non-therapy application, distinguishable from a therapy application in that the therapy application can be used to treat a disorder such as a communication disorder, and the non-therapy application can be used to assist with and/or alleviate the disorder such as to allow the patient to communicate with at least one of their own speech or audio communications generated by system 10. Examples of a non-therapy application include: a picture-based electronic communication tool; an audio-based electronic communication tool; a game such as a video game; a news information reader; a telephone internet program; a location-based application using GPS information to provide stimuli for various purposes such as information review; a provision of utterance feedback; a stimuli to cue functional speech and the like; and combinations of these. Data input to user input assembly 110 can be used to control the non-therapy applications.

System 10 can include a patient health monitor, not shown but configured to detect one or more patient physiologic parameters and/or speech or motor functions indicative of an adverse event such as a patient stroke. The health monitor may analyze data input into user input assembly 110, such as audio, video and/or motor-function related input data. For example, if a patient is performing a therapy application where the therapy application requires the patient to speak, and the patient begins to slur his or her words, system 10 can detect the change in the patient's speech and alert an emergency service provider. Also, system 10 can be constructed and arranged to notify any or all of the patient's therapists, caregivers, and/or family members.

System 10 can include a one-click emergency button configured to allow a user to contact an emergency contact, such as any or all of the patient's therapists, caregivers, family members, and/or an emergency service provider. For example, each screen of a therapy application can include the one-click emergency button.

System 10 can include a remote control function, for example to allow a representative of the manufacturer to remotely perform (e.g. at a location remote from the patient) a function selected from the group consisting of: log in to a system component; control a system component; troubleshoot a system component; modify a system component; train a patient; and combinations of these. Also within this function or via a separate function, system 10 can be constructed and arranged to transfer control and/or information from a first user to a second user. For example, where the first user is a patient's SLP and the second user is the same patient's physical therapist, the two therapists can transfer any information regarding the patient in order to optimize two separate therapies or a combination of therapies. Additionally, both therapists can control and/or access the patient's data and results. The two users can be any combinations of users, users described herein, and more than two users can utilize this function, for example any or all of the patient's therapists can access, control and transfer patient data or results.

System 10 can further include a login function configured to allow a user to access the system by entering a username and/or a password. The login function can be configured to allow multiple levels of authorization based on the username. For example, a therapist can access multiple patients' accounts that are linked with their particular username.

System 10 can include an email function enabling any user, examples of users described herein, to send an email, such as an email sent to any or all users. Additionally, system 10 can be constructed and arranged to allow any representative of the manufacturer of the system to email any user.

System 10 can include a billing algorithm configured to facilitate the billing of users of the system and/or a third party responsible for the user's payment (e.g. an insurance company, family member, etc). For example, system 10 can employ a pay per use algorithm and/or a pay per time period algorithm. Optionally, the algorithm can include a discount based on at least one of: user feedback; extended personal information provided by a user; or user assessments.

System 10 can further include an external input assembly configured to receive external data from an external source, for example via a wire; the internet; a wireless connection such as Bluetooth or cellular service connection; and combinations of these. Examples of external data include: medical data such as medical statistics, medical references, patient or other user group data; medical encyclopedias, and the like; user billing data; local or world news; or any other data available via the internet.

System 10 can be configured to run in various modes, for example one or more multiple user modes that allow two or more users to participate in a therapy application. Examples of the modes include: family mode; multiple patient mode; multiple caregiver mode; and combinations of these. Details regarding the various modes are described in FIG. 4 herebelow.

Alternative to or in addition to treating a communication disorder, system 10 can be used to provide another benefit to the patient, such as to act as an electronic communicator. System 10 can be constructed and arranged to provide a benefit selected from the group consisting of: a therapeutic benefit; an orthotic benefit; a prosthetic benefit; and combinations of these.

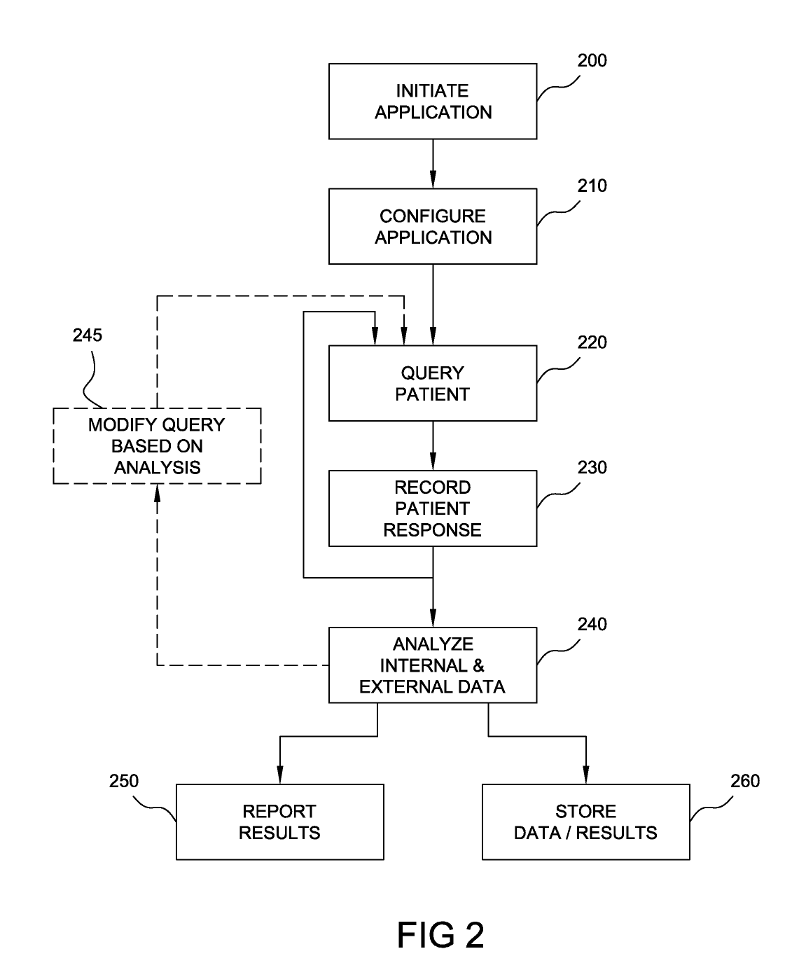

FIG. 2 illustrates a method for treating a communication disorder, consistent with the present inventive concepts. The illustrated method can be carried out by a system such as system 10 described in FIG. 1 hereabove. In STEP 200, an application is initiated, such as a therapy application described in reference to FIG. 1 hereabove. The application can be initiated by a user, in this example the user is a patient, for example if the patient is using the system at his or her own home. Additionally or alternatively, another user of the system can initiate the application. Other applicable users include but are not limited to: a therapist, a family member, a second patient; a support group member; and combinations of these. Application initiation can be performed locally or via the system's remote control function, described in FIG. 1 hereabove. In some embodiments, STEP 200 is performed after a diagnostic procedure is performed, for example where the type or configuration of the application can be chosen and/or modified, and then initiated based on the type of communication disorder with which the patient is diagnosed. Details of a diagnostic procedure are described in FIG. 3 herebelow.

In STEP 210, the application can be customized. The therapy applications and/or the user interface can be customizable by a user, such as a patient or a therapist. For example, a therapy application can include multiple user selectable levels, for example “easy”, “medium” and “hard” levels. The therapy application can include multiple user selectable therapy sub-applications, for example where a sub-application includes content such as motion picture content; trivia content; sports information content; historic information content; and combinations of these. Additionally or alternatively, the therapy application can include multiple user selectable therapy sub-applications, where each sub-application includes a different functionality. Examples of applicable functions include but are not limited to: mail function such as an email function; phone function such as internet phone function; news retrieval function; word processing function; accounting program function such as a bill paying function; video or other game playing function; and combinations of these. Additionally or alternatively, the therapy application can include multiple user selectable therapy sub-applications, where each sub-application provides different information to the patient such as icons displayed; pictures displayed; text displayed; audio provided; moving video provided; and combinations of these. In one example, the user (e.g. the patient) can customize the therapy application by selecting the content and functionality of the application and/or interface based on personal preference and/or area of improvement needed. For example, if the patient is at a beginner level and is an animal lover, the patient can select an “easy” level therapy application with content such as icons, pictures, audio, text and/or moving video that is animal based.

The customization and/or any modification of the user interface and/or therapy application can be manual or automatic based upon one or more parameters or upon data collected through the performance of one or more procedures. For example, the interface and/or application can adapt based upon a diagnostic procedure and/or a user's performance of the therapy application or other therapy application, details of which are described in FIGS. 3 and 5 herebelow.

In STEP 220, the patient is queried. For example, the therapy application can include one or more questions requiring one or more answers from the patient. As an example, the therapy application can include a user interface displaying five pictures of five different animals, and the therapy application can query the patient to identify which icon or picture represents a dog where the patient can respond, for example by clicking on the icon and/or touching a presented icon. A series of questions similar or dissimilar to one another can be provided to the patient.

In STEP 230, the patient response is recorded. Continuing with the above example, the patient can then select the correct answer (i.e. the picture that represents the dog), or the patient can select an incorrect answer (i.e. a picture that represents a cat or other non-dog icon). This response, as well as any or all of the responses included in the therapy application, is recorded by the system and can be stored in a data library, such as data library 140 of FIG. 1 hereabove. Then, the method repeats at STEP 220, where the patient is queried again until the therapy application is complete.

Once the therapy application is complete, STEP 240 is performed where data is analyzed, for example via a data analyzer such as data analyzer 121 of FIG. 1. Examples of data include internal data such as patient responses in the form of: recorded speech, such as speech representing at least one of a sentence; a word; a partial word; a phonetic sound such as a diphone, a triphone or a blend; a phoneme; a syllable; written data; patient movement data such as lip or tongue movement data; patient physiologic data; patient psychological data; patient historic data; and combinations of these. Examples of written data include: written data generated by the patient, such as data selected from the group consisting of: a picture; text; written words; icons; symbols; and combinations of these. Examples of patient movement data include: data recorded from a video camera; data recorded from a keypad or keyboard entry; data recorded from mouse movement or clicking; eye movement data; and combinations of these. Examples of patient physiologic data include: Meyer-Briggs personality structure data; Enneagram type data; diagnostic and other data related to disorders such as cognitive disorders and memory disorders; data representing instances of depression; and combinations of these. Examples of patient historic data include: patient previous surgery or illness data; family medical history; and combinations of these. Additionally, external data can be analyzed including data such as medical reference data such as medical statistics from one or more similar patient populations; medical literature relevant to the patient disorder; and combinations of these.

In some embodiments, the analysis of STEP 240 includes a manual analysis step that can be performed, for example, by a therapist at a location remote from the patient as the patient is using the system (i.e. in real time). Alternatively, the manual analysis can be performed at a time after data has been entered by the patient, such as a time within one hour from the time the data was entered; more than four hours after the data was entered; or more than twenty-four hours after the data was entered. In some embodiments, the analysis is a fully automated or at least a partially automated analysis of the data, for example where the system further includes an automated speech language pathologist function configured to perform any or all functions of an SLP, described in detail in FIG. 1 hereabove. In some embodiments, the analysis can include at least one manual analysis step and at least one automated analysis step. The analysis can include a quantitative and/or a qualitative analysis of the data.

Based on the analysis, an optional STEP 245 can be performed where the therapy application or any portions thereof can be modified. Therapy modifications can include a change in content and/or functionality. For example, a content change can include a change to the user interface. A content change can include a change to a therapy difficulty level, for example “easy”, “medium” and “hard” levels. A content change can include a change to a sub-application, for example where a sub-application includes content such as motion picture content; trivia content; sports information content; historic information content; and combinations of these. A content change can include a change between therapy sub-applications, where each sub-application includes a different functionality. Examples of functions that may be changed include: phone function such as internet phone function; news retrieval function; word processing function; accounting program function; and combinations of these. Additionally or alternatively, multiple user selectable therapy sub-applications can be changed, where each sub-application provides different information to the patient such as icons displayed; pictures displayed; text displayed; audio provided; moving video provided; and combinations of these. In one example, the user (e.g. the patient) can change the therapy application by selecting the content and functionality of the application and/or interface based on personal preference and/or area of improvement needed. In one example, a patient changes from a beginner level to a more advanced level. In another example, the patient changes the genre content of the material, such as from sports to history.

When the modification of the therapy application is complete, the method repeats, beginning at STEP 220. STEP 220 through STEP 245 can be repeated until the therapy application includes suitable content and functionality for a particular patient, such as after successful results are achieved, after a time period has elapsed and/or after another event has occurred.

IN STEP 250, results can be generated and reported. A report generator can be configured to provide one or more results to a user, such as report 131 described in reference to FIG. 1 hereabove. The results can be displayed, for example via a visual display; a touchscreen; a speaker; a tactile transducer; a paper printout generated by a printer; and combinations of these. The report can be similar and/or correlate to a standard communication disorder assessment test report, for example, assessments such as scientific analyses of data gathered using WHO taxonomy of disease such as impairment, activity limitation, and participation restriction; WAB; BDAE; PICA; BNT; PNT; ASHA-FACS; ASHA-QOCL; other standardized assessments of aphasia; and data from non-standardized assessment instruments such as the PALPA. The report can display results in a variety of ways, for example via a graphical representation; a representation of percentages; a representation of comparisons; and combinations of these. Examples of comparisons include: comparison of results from the same patient; comparison of results from the patient and a different patient; comparison of results from the patient to a summary of multiple patient data; and combinations of these.

In STEP 260, any or all data, results, and/or reports generated during the performance of the illustrated method can be stored, for example in a data library such as data library 140 of FIG. 1 hereabove.

Alternative to or in addition to treating a communication disorder, the illustrated method of FIG. 2 can serve as a communication tool. The method of FIG. 2 can be performed to provide numerous benefits to the patient, such as a benefit selected from the group consisting of: a therapeutic benefit; an orthotic benefit; a prosthetic benefit; and combinations of these.

FIG. 3 illustrates a method for diagnosing a patient with a communication disorder, consistent with the present inventive concepts. The illustrated method can be carried out by a system such as system 10 described in FIG. 1 hereabove. In STEP 300, one or more patient diagnostics are run. The patient diagnostic can include a customized therapy application or a standardized or non-standardized test configured to generate diagnostic data to facilitate a patient diagnosis and/or prognosis. Examples of standardized tests include: scientific analyses of data gathered using WHO taxonomy of disease such as impairment, activity limitation, and participation restriction; WAB; BDAE; PICA; BNT; PNT; ASHA-FACS; ASHA-QOCL; other standardized assessments of aphasia; and data from non-standardized assessment instruments such as the PALPA.

In STEP 310, the diagnostic data is analyzed, for example via a data analyzer such as data analyzer 121 of FIG. 1. This analysis step can include a manual analysis step that can be performed by, for example, a therapist at a location remote from the patient at some time after data is entered such as a time within one hour from the time the data is entered; more than four hours after the data is entered; or more than twenty-four hours after the data is entered. In some embodiments, the analysis is a fully automated, or at least a partially automated analysis of the data for example where the system further includes an automated speech language pathologist function configured to perform any or all functions of an SLP, described in detail in FIG. 1 hereabove. In some embodiments, the analysis can include at least one manual analysis step and at least one automated analysis step. The analysis can include a quantitative and/or a qualitative analysis of the data.

In STEPs 320, 330, 340, 350, and 360, the patient's speech, comprehension, reading skills, writing skills, and any other ability that may be generally relevant or specific to the patient, respectively, are evaluated and analyzed. Other abilities evaluated and analyzed in STEP 360 can include memory, inference, and judgment, as an example. These steps can be performed in any order, and any or all of the steps can be removed from the diagnostic procedure based upon the particular patient. One or more of these steps can be repeated, such as when an unsatisfactory result or inadequate recording is detected, such as by system 10 of FIG. 1. These steps can be performed via a therapy application; a standardized or non-standardized test; or any other exercise to satisfactorily evaluate the particular ability.

Any of the data acquired in STEPs 300-360 can produce results and/or reports, examples of which are described in detail with reference to FIG. 1 hereabove.

In STEP 370, the patient diagnosis is compiled. Based on an analysis of the data acquired in STEPs 300-360, the type of communication disorder can be determined. In addition, other assessments can be made, for example, future disease state status; disease state progression; expected improvement after using the therapy application; a comparison of the expected improvement to be achieved with a first therapy application with the expected improvement to be achieved with a second, different therapy application; prognostic indications from scientific analyses of data gathered using WHO taxonomy of disease such as impairment, activity limitation, and participation restriction; WAB; BDAE; PICA; BNT; PNT; ASHA-FACS; ASHA-QOCL; other standardized assessments of aphasia; data from non-standardized assessment instruments such as the PALPA; and combinations of these.

FIG. 4 illustrates a system for treating a communication disorder and configured to allow multiple user input, consistent with the present inventive concepts. System 10′ can be constructed and arranged to record data from a single user or multiple users, where a user can include an SLP; a patient; a second patient; a representative of the system manufacturer; a family member of the patient; a support group member; a clinician; a nurse; a caregiver; a healthcare statistician; a hospital; a healthcare insurance provider; a healthcare billing service; and combinations of these.

In the illustrated embodiment, first user 401, second user 402, and third user 403 through the “n^{th}” user 404 are participating in a therapy application. In a first example, all users are patients, for example where system 10′ is operating in a multiple patient mode. In this mode, some or all patients can perform the same therapy application, or each patient can perform a different application, for example a personally customized therapy application as has been described herein. If all patients are performing the same therapy application, they can be performing the therapy application at different times or at the same time, either interactively or independently.

Each user enters data into system 10′, such as via one or more input devices as are described in reference to system 10 of FIG. 1 hereabove. Data entered can include data related to the performance of a therapy application, for example recorded speech, such as speech representing at least one of: a sentence; a word; a partial word; a phonetic sound such as a diphone, a triphone or a blend; a phoneme; or a syllable. Additionally, written data; patient movement data such as lip or tongue movement data; patient physiologic data; patient psychological data; patient historic data; and combinations of these can be entered. Examples of written data include written data generated by the patient, such as data selected from the group consisting of: a picture; text; written words; icons; symbols; and combinations of these. Examples of patient movement data include: data recorded from a video camera; data recorded from a keypad or keyboard entry; data recorded from mouse movement or clicking; eye movement data; and combinations of these. Examples of patient physiologic data include: Meyer-Briggs personality structure data; Enneagram type data; diagnostic and other data related to disorders such as cognitive disorders and memory disorders; data related to instances of depression; and combinations of these. Examples of patient historic data include: patient previous surgery or illness data; family medical history; and combinations of these.

The data described in the paragraph above can be filtered by data filter 410, where data can be filtered according to each user, or similar data from each user can be filtered and combined so that an analysis can be performed by data analyzer 420, for example a comparison of similar data entered by any or all users. Data can be filtered and/or combined in any useful way so that data analyzer 420 can then analyze the data.

Data analyzer 420 can be configured to analyze the filtered data and produce results based on the analysis and can include a similar construction and functionality as data analyzer 121 of FIG. 1. In the case of multiple patient users, data analyzer 420 can be configured to produce results based on any combination of results from user 401 through user 404. Subsequently, a therapy application can be selected and/or an existing therapy application can be modified for any or all patients based on the combined results.

System 10′ can also be configured to run in other modes, for example family mode; multiple caregiver mode; and combinations of these. In family mode, user 401 can include a patient and users 402 through 404 can include family members, where all users 401 through 404 can interactively perform the same therapy application, for example so as to assist, motivate, evaluate, and/or monitor the patient in his or her performance of the therapy application. In a multiple caregiver mode, user 401 can include a patient and users 402 through 404 can include different caregivers, for example where user 402 is an SLP, user 403 is a physical therapist, and user 404 is a psychologist. In this example, all users 401 through 404 can interactively perform the same therapy application, for example so as to assist, evaluate, and/or monitor the patient in his or her performance of the therapy application.

Data entered by any or all users, filtered data, and any analysis and/or results generated from data analyzer can be stored in data storage 430. This data can be accessed and utilized by any or all users of system 10′, or the data can be protected, for example so that only certain users can access (.e.g. view and/or modify) certain data.

FIG. 5 illustrates a method for determining the appropriate level of therapy for a patient with a communication disorder, consistent with the present inventive concepts. In STEP 500 patient data is analyzed. Examples of patient data include data related to the performance of a therapy application. In some embodiments, data includes recorded speech, such as speech representing at least one of: a sentence; a word; a partial word; a phonetic sound such as a diphone, a triphone or a blend; a phoneme; or a syllable. Additionally, written data; patient movement data such as lip or tongue movement data; patient physiologic data; patient psychological data; patient historic data; and combinations of these can be entered. Examples of written data include written data generated by the patient selected from the group, such as data selected from the group consisting of: a picture; text; written words; icons; symbols; and combinations of these. Examples of patient movement data include: data recorded from a video camera; data recorded from a keypad or keyboard entry; data recorded from mouse movement or clicking; eye movement data; and combinations of these. Examples of patient physiologic data include: Meyer-Briggs personality structure data; Enneagram type data; diagnostic and other data related to disorders such as cognitive disorders and memory disorders; instances of depression; and combinations of these. Examples of patient historic data include: patient previous surgery or illness data; family medical history; and combinations of these. Additionally, data from multiple patients can be included in the analysis, for example as has been described in FIG. 4 hereabove. Further, external data such as medical reference data such as medical statistics; medical literature relevant to the patient disorder; and the like can be included in the analysis.

The analysis can be performed by a data analyzer of a system, for example system data analyzer 121 of system 10 and/or data analyzer 420 of system 10′ described herein such that the analysis generates results. Additionally or alternatively, the patient data can be analyzed by any or all of the patient's caregivers, therapists, or family members.

In STEP 510, based on the results generated from the analysis in STEP 500, the system can determine whether or not a therapy should be changed, and if so, how the therapy should be changed. For example, if the user, e.g. the patient, has been receiving a consistently high score on a particular therapy application, the system can identify this occurrence. In addition to or alternative to the system identifying a reason for a therapy change, any or all users of the system, e.g. the patient's caregivers, therapists, or family members can make this determination. Some other considerations in determining if a therapy change is appropriate can include: the cumulative results of the multiple therapy applications; the averaged results of the multiple therapy applications; the standard deviation of the results of the multiple therapy applications; a trending analysis of the results of the multiple therapy applications; and combinations of these. Additionally or alternatively, a procedure similar to the diagnostic procedure described in FIG. 3 can be performed to assist in the determination.

In STEP 520, the system queries the user (e.g. patient) and/or any or all of the patient's caregivers, therapists, or family members, asking if the change should be implemented. For example, if the user has been receiving consistently high scores on a therapy application at the “easy” level, the system can prompt the user if he or she would like to change to the “medium” difficulty level. In addition to or alternative to the system prompting the user, any or all users of the system (e.g. the patient's caregivers, therapists, or family members) can make this request and/or determination.

If the therapy change is not approved by any or all users, the change will not be implemented, as shown in STEP 530. However, if the change is approved by any or all users, the change will be implemented, as shown in STEP 540. Therapy changes can include a change in content and/or functionality. For example, a content change can include a change to at least one of: the user interface; a therapy application; a therapy sub-application or theme; a therapy difficulty level; a system function such as a phone or financial function; or a multiple user parameter, all as have been described hereabove.

In the example where the therapy change is approved and implemented by a user other than the patient (e.g. caregivers, therapists, or family members), a remote control function can be used. For example the user can: log in to a system component such as via a wired or wireless connection; control a system component; control and/or access the patient's account; and combinations of these.

If a therapy change is not implemented, STEPs 500 through 540 can be repeated. Likewise, if a change is implemented, the method can be repeated to determine if any other change(s) should be implemented.

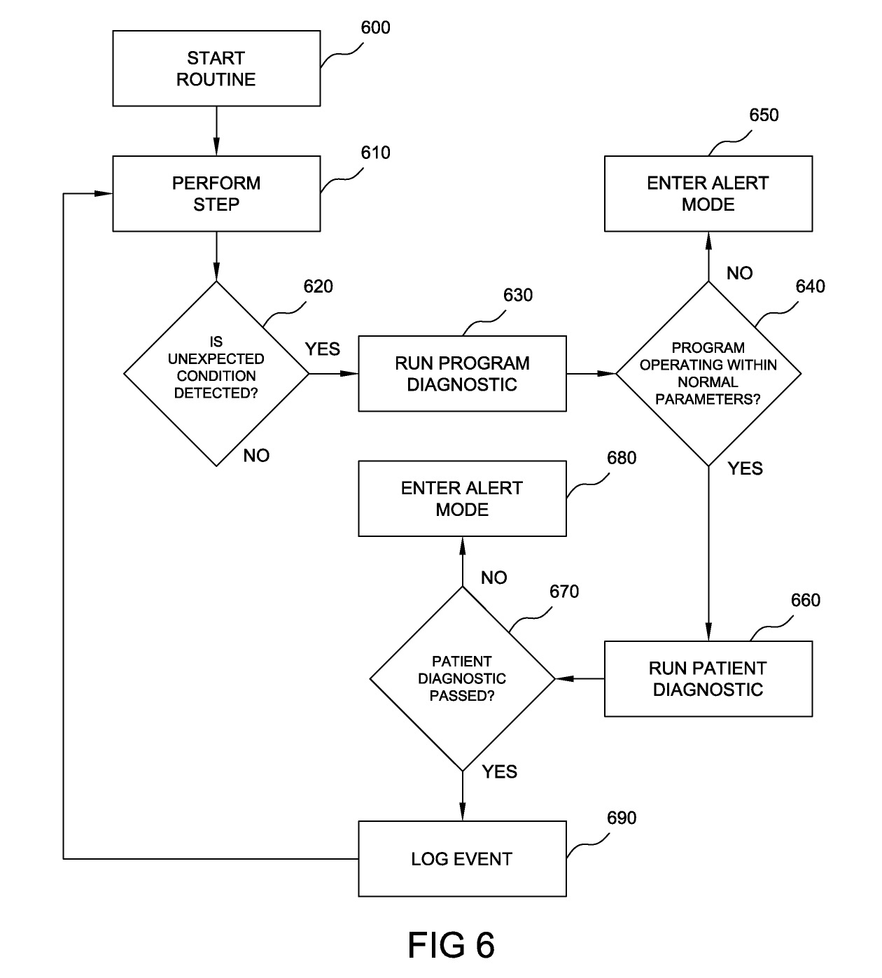

FIG. 6 illustrates a self-diagnostic algorithm, consistent with the present inventive concepts. A system, for example system 10 of FIG. 1 or system 10′ of FIG. 4 can include a diagnostic assembly employing a self diagnostic algorithm. The algorithm can include a software algorithm that can be completely or partially automated to determine if a therapy application or other application of the system is operating within its parameters and/or thresholds. The self-diagnosing algorithm includes both an analysis of the system via a system diagnostic and the user via a patient diagnostic. The algorithm can be run via an assembly, for example a hardware assembly. In STEP 600, the algorithm is initiated to identify any unexpected or adverse conditions.

In STEP 610, the algorithm searches for unexpected conditions. This step can include other algorithmic functions, for example, a mute detection algorithm can be employed to detect an inadvertent mute condition of a system audio component, such as a microphone that records user spoken data.

In STEP 620, if applicable, the algorithm detects an unexpected condition. Continuing with the mute detection example, the algorithm can detect if no audio has been recorded for a period of time, where a threshold for the acceptable period of time can be set by any user of the system. If the microphone is functioning properly, then the routine is complete. However, if the algorithm detects that a therapy application is being performed, but no audio has been recorded for a period of time, for example 30 minutes, then a system diagnostic can be performed, as shown in STEP 630.

In STEP 640, the algorithm will determine if one or more microphones are functioning properly. If a microphone is not functioning properly, the system can enter an alert mode, as shown in STEP 650. The alert mode can be configured to notify any or all users of the system and manufacturers of the system that the microphone requires maintenance and/or needs to be replaced.

In the case where one or more microphones are functioning properly, the algorithm will perform STEP 660 and run a patient diagnostic. In this step, the algorithm can query the user, for example an audio or a text query asking the user to speak or click “OK” if he or she is performing the application. In STEP 670, if the patient responds, the system logs the event as shown in STEP 690 indicating the diagnostic was successfully run and no unexpected or adverse conditions were detected, and the method is repeated. Any or all events occurring during the performance of this illustrated method can be logged into the system, for example in a data library, such as data library 140 of FIG. 1.

If the user does not respond within a particular period of time, where the length of time can be preset by any user of the system, an alert mode is entered, as shown in STEP 680. Here, the alert mode can include contacting an emergency service provider such as the police, fire department or an ambulance service. Additionally or alternatively, the user's therapist; caregiver; family member; manufacturer of the system; or the like can be contacted.

Other examples of unexpected conditions identified by the system of the present inventive concepts include: power failure; inadequate signal level such as inadequate signal level recorded at the user input assembly; interruption in data gathering; factors affecting human performance such as distraction, patient or other operator fatigue, patient or other operator aggravation; interruption in data transmission; unexpected system failure; unexpected termination of a user task; and combinations of these.

A mute detection algorithm was used as an example of the illustrated method, however many algorithms can be employed to determine an unexpected system and/or user condition. For example, a threshold algorithm can be employed that is configured to compare a parameter to a threshold. Examples of applicable parameters include but are not limited to: lexical parameters such as word length, number of syllables, segmental challenge, phonotactic challenge, abstractness, and age of acquisition; syntactic parameters such as mean length of utterance, phrase structure complexity, and ambiguity metrics; pragmatic parameters such as contextual interpretation support, and salience for particular patient; number or percentage of incorrect answers; number or percentage of correct answers; time taken to perform a task; user input extraneous to completing a task; period of inactivity; time between user input events; hesitation pattern analysis; and combinations of these. If a parameter is outside of a threshold, the system can enter an alert mode, as discussed hereabove.

Another self-diagnostic algorithm example includes a time-out function that is configured to detect a pre-determined level of user inactivity from all inputs of the system, including: microphone; mouse; keyboard; touchscreen; camera; eye tracking device; joystick; trackpad device; sip and puff device; gesture tracking device; brain interface machine; any computer input device; and combinations of these. If the system detects inactivity on any or all of these inputs for a particular period of time, the system can enter an alert mode, as discussed hereabove. In some embodiments, a threshold of inactivity comprises 30 seconds, 1 minute, 5 minutes, 10 minutes or 15 minutes.

The foregoing description and accompanying drawings set forth a number of examples of representative embodiments at the present time. Various modifications, additions and alternative designs will become apparent to those skilled in the art in light of the foregoing teachings without departing from the spirit hereof, or exceeding the scope hereof, which is indicated by the following claims rather than by the foregoing description. All changes and variations that fall within the meaning and range of equivalency of the claims are to be embraced within their scope.

We claim:

1. A system for a patient, comprising:
a user input assembly constructed and arranged to allow a user to enter input data;
an input data analyzer constructed and arranged to receive the input data, analyze the input data and produce results based on the input data;
a user output assembly constructed and arranged to receive the results from the input data analyzer and display the results; and
a data library constructed and arranged to receive the results from the input data analyzer and store the results.

2. The system of claim 1 wherein the system is constructed and arranged to treat a patient having a communication disorder comprising a disorder selected from the group consisting of: aphasia; apraxia of speech; dysarthria; dysphagia; and combinations thereof.

3. The system of claim 2 wherein aphasia is selected from the group consisting of: global aphasia; isolation aphasia; Broca's aphasia; Wernicke's aphasia; transcortical motor aphasia; transcortical sensory aphasia; conduction aphasia; anomic aphasia; primary progressive aphasia; and combinations thereof.

4. The system of claim 2 wherein the system is further constructed and arranged to treat a condition selected from the group consisting of: conditions of motor involvement such as right hemiplegia; sensory involvement such as right hemianopsia and altered acoustic processing; cognitive involvement such as memory impairments, judgment impairments and initiation impairments; and combinations thereof.

5. The system of claim 2 wherein the communication disorder comprises a disorder caused by at least one of: a stroke; a trauma to the brain or a congenital disorder; a medical accident or side effect thereof; a traumatic brain injury; a penetrating head wound; a closed head injury; a tumor; a medical procedure adverse event; an adverse effect of medication.

6. The system of claim 1 wherein the system is constructed and arranged to provide a therapeutic benefit to the patient.

7. The system of claim 1 wherein the system is constructed and arranged to provide an orthotic benefit to the patient.

8. The system of claim 1 wherein the system is constructed and arranged to provide a prosthetic benefit to the patient.

9. The system of claim 1 wherein the user input assembly comprises an assembly selected from the group consisting of: microphone; mouse; keyboard; touchscreen; camera; eye tracking device; joystick; trackpad; sip and puff device; gesture tracking device; brain machine interface; any computer input device; and combinations thereof.

10. The system of claim 1 wherein the user comprises a user selected from the group consisting of: a speech language pathologist; the patient; a second patient; a representative of the system manufacturer; a family member of the patient; a support group member; a clinician; a nurse; a caregiver; a healthcare statistician; a hospital; a healthcare insurance provider; a healthcare billing service; and combinations thereof.

11. The system of claim 1 wherein the user comprises multiple users.

12. The system of claim 11 wherein the multiple users comprise at least the patient and a speech and language pathologist.

13. The system of claim 11 wherein the multiple users comprise at least the patient and a second patient.

14. The system of claim 1 wherein the input data comprises at least recorded speech.

15. The system of claim 14 wherein the recorded speech comprises speech representing at least one of: a sentence; a word; a partial word; a phonetic sound such as a diphone, a triphone or a blend; a phoneme; or a syllable.

16. The system of claim 14 wherein the input data further comprises data selected from the group consisting of: written data; patient movement data such as lip or tongue movement data; patient physiologic data; patient psychological data; patient historic data; and combinations thereof.

17. The system of claim 1 wherein the input data comprises at least patient written data.

18. The system of claim 17 wherein the patient written data comprises data generated by the patient selected from the group consisting of: a picture; text; written words; icons; symbols; and combinations thereof.

19. The system of claim 1 wherein the input data comprises at least patient movement data.

20. The system of claim 19 wherein the patient movement data comprises data selected from the group consisting of: data recorded from video camera; data recorded from keypad or keyboard entry; data recorded from mouse movement or clicking; eye movement data; and combinations thereof.

21. The system of claim 1 wherein the input data comprises at least patient physiologic information.

22. The system of claim 21 wherein the patient physiologic information is selected from the group consisting of: monitored galvanic skin response; respiration; EKG; visual information such as left field cut and right field cut; macular degeneration; lack of visual acuity; limb apraxia; limb paralysis; and combinations thereof.

23. The system of claim 1 wherein the input data comprises at least patient psychological information.

24. The system of claim 23 wherein the patient psychological information is selected from the group consisting of: Meyer-Briggs personality structure data; Enneagram type data; diagnostic and other data related to disorders such as cognitive disorders and memory disorders; data representing instances of depression; and combinations thereof.

25. The system of claim 1 wherein the input data comprises at least patient historic data.

26. The system of claim 25 wherein the patient historic data comprises data selected from the group consisting of: patient previous surgery or illness data; family medical history; and combinations thereof.

27. The system of claim 1 wherein the input data comprises at least external data.

28. The system of claim 27 wherein the external data comprises data selected from the group consisting of: medical reference data such as medical statistics from one or more similar patient populations; medical literature relevant to the patient disorder; user billing data; local or world news; any data available via the internet; and combinations thereof.

29. The system of claim 1 wherein the user output assembly comprises an output assembly selected from the group consisting of: a visual display; a touchscreen; a speaker; a tactile transducer; a printer for generating a paper printout; and combinations thereof.

30. The system of claim 1 wherein the data analyzer analysis comprises a manual analysis step performed by an operator.

31. The system of claim 30 wherein the system is constructed and arranged such that the manual analysis step is performed at a location remote from the patient.

32. The system of claim 31 wherein the system is constructed and arranged such that the manual analysis step is performed at approximately the same time that the input data is entered.

33. The system of claim 31 wherein the system is constructed and arranged such that the manual analysis step is performed within one hour that the input data is entered.

34. The system of claim 31 wherein the system is constructed and arranged such that the manual analysis step is performed at least four hours after the input data is entered.

35. The system of claim 31 wherein the system is constructed and arranged such that the manual analysis step is performed at least twenty-four hours after the input data is entered.

36. The system of claim 1 wherein the data analyzer analysis comprises an at least partially automated analysis.

37. The system of claim 36 wherein the data analyzer analysis comprises a fully automated analysis.

38. The system of claim 36 wherein the data analyzer analysis comprises at least one manual analysis step and at least one automated analysis step.

39. The system of claim 1 wherein the data analyzer analysis comprises a quantitative analysis.

40. The system of claim 1 wherein the results comprise a status of the patient's disease state.

41. The system of claim 40 wherein the status of the patient's disease is assessed via at least one of: involvement indications and profiles from WHO taxonomy of disease such as impairment, activity limitation, and participation restriction; WAB; BDAE; PICA; BNT; PNT; ASHA-FACS; ASHA-QOCL; and other standardized or non-standardized assessments of aphasia and other language disorders, language impairment, functional communication, satisfaction, and quality of life.

42. The system of claim 1 wherein the results comprise an assessment of improvement of the patient's disease state.

43. The system of claim 42 wherein the improvement of the patient's disease is assessed via at least one of: improvements as revealed by statistical analyses of assessments from WHO taxonomy of disease such as impairment, activity limitation, and participation restriction; WAB; BDAE; PICA; BNT; PNT; ASHA-FACS; ASHA-QOCL; other standardized assessments of aphasia; and data from non-standardized assessment instruments such as the PALPA.

44. The system of claim 1 wherein the results comprise an assessment of a patient therapy.

45. The system of claim 44 further comprising a therapy application constructed and arranged to provide the patient therapy.

46. The system of claim 1 wherein the results comprise an assessment of a first patient therapy and a second patient therapy.

47. The system of claim 46 further comprising a therapy application constructed and arranged to provide at least the first patient therapy.

48. The system of claim 1 wherein the results comprise an assessment of a patient parameter selected from the group consisting of: functional communication level; speech impairment level; quality of life; impairment, activity limitation; participation restriction; and combinations thereof.

49. The system of claim 1 wherein the results comprise a patient prognosis.

50. The system of claim 49 wherein the prognosis comprises a prognosis of at least one of future disease state status; disease state progression; prognostic indications from scientific analyses of data gathered using WHO taxonomy of disease such as impairment, activity limitation, and participation restriction; WAB; BDAE; PICA; BNT; PNT; ASHA-FACS; ASHA-QOCL; or other standardized or non-standardized assessments of aphasia.

51. The system of claim 49 further comprising a therapy application, wherein the prognosis comprises an estimation of expected improvement after using the therapy application.

52. The system of claim 49 wherein the system comprises a first therapy application and a second therapy application, wherein the prognosis compares the expected improvement to be achieved with the first therapy application with the expected improvement to be achieved with the second therapy application.

53. The system of claim 1 wherein the results comprise a report similar to a standard communication disorder assessment test report.

54. The system of claim 53 wherein the standardized assessment of the communication disorder is selected from the group consisting of: scientific analyses of data gathered using WHO taxonomy of disease such as impairment, activity limitation, and participation restriction; WAB; BDAE; PICA; BNT; PNT; ASHA-FACS; ASHA-QOCL; and other standardized or non-standardized assessments of aphasia.

55. The system of claim 1 wherein the results comprise a report which can be correlated to a standard communication disorder assessment test report.

56. The system of claim 55 wherein the report is generated via at least one of: scientific analyses of data gathered using WHO taxonomy of disease such as impairment, activity limitation, and participation restriction; WAB; BDAE; PICA; BNT; PNT; ASHA-FACS; ASHA-QOCL; and other standardized or non-standardized assessments of aphasia.

57. The system of claim 1 wherein the results comprise a playback of recorded data.

58. The system of claim 57 wherein the recorded data comprises data selected from the group consisting of: video recordings, audio recordings, movement data such as keystroke entry data; and combinations thereof.

59. The system of claim 58 wherein the recorded data playback is manipulated by at least one of: a fast forward manipulation; a rewind manipulation; a play manipulation; a stepped-playback manipulation; or a pause manipulation.

60. The system of claim 1 wherein the results comprise a summary of keystrokes.

61. The system of claim 60 wherein the summary of keystrokes comprises a summary of keystrokes made by at least the patient.

62. The system of claim 60 wherein the summary of keystrokes comprises a summary of keystrokes made by at least a non-patient user of the system.

63. The system of claim 1 wherein the results comprise at least a numerical score.

64. The system of claim 1 wherein the results comprise at least a qualitative score.

65. The system of claim 1 wherein the results comprise a representation of a pattern of error.

66. The system of claim 1 wherein the results comprise an analysis of a patient's speech.

67. The system of claim 1 wherein the results comprise an analysis of patient written data.

68. The system of claim 1 wherein the results comprise an analysis of patient answer choices.

69. The system of claim 1 wherein the results comprise an analysis of at least one of: keystrokes; mouse clicks; body movement such as lip, tongue or other facial movement; or touch screen input.

70. The system of claim 1 further comprising a therapy application, wherein the results comprise an analysis of at least one of a time duration of the therapy application; or elapsed time between segments of the therapy application.

71. The system of claim 1 further comprising a therapy application.

72. The system of claim 71 wherein the therapy application comprises multiple user selectable levels.

73. The system of claim 72 wherein the multiple levels comprise at least a first level; a second level more difficult than the first level; and a third level more difficult than the second level.

74. The system of claim 71 wherein the therapy application comprises multiple user selectable therapy sub-applications.

75. The system of claim 74 wherein a first therapy sub-application comprises different content than a second therapy sub-application.

76. The system of claim 75 wherein the first therapy sub-application content comprises content selected from the group consisting of: motion picture content; trivia content; sports information content; historic information content; and combinations thereof.

77. The system of claim 74 wherein a first therapy sub-application comprises different functionality than a second therapy sub-application.

78. The system of claim 77 wherein the first therapy functionality comprises a function selected from the group consisting of: mail function such as an email function; phone function such as internet phone function; news retrieval function; word processing function; accounting program function such as bill paying function; video or other game playing function; and combinations thereof.

79. The system of claim 74 wherein a first therapy sub-application comprises different patient provided information than a second therapy sub-application.

80. The system of claim 79 wherein the different patient provided information comprises a difference in at least one of: icons displayed; pictures displayed; text displayed; audio provided; or moving video provided.

81. The system of claim 79 wherein the first therapy sub-application comprises different patient provided information than the second therapy sub-application based on an adaptive signal processing of ongoing user performance.

82. The system of claim 71 wherein the therapy application comprises an application selected from the group consisting of: a linguistic therapy application; a syntactic therapy application; an auditory comprehension therapy application; a reading therapy application; a speech production therapy application; a cognitive therapy application; a cognitive reasoning therapy application; a memory therapy application; a music therapy application; a video therapy application; a lexical therapy application exercising items such as word length, number of syllables, segmental challenge levels, phonotactic challenge levels, word frequency data, age of acquisition data, iconic transparency, and iconic translucency; and combinations thereof.

83. The system of claim 71 wherein the therapy application comprises a therapy application based on a patient diagnostic evaluation.

84. The system of claim 71 wherein the therapy application comprises a therapy application based on a communication disorder diagnosed for the patient.

85. The system of claim 71 wherein the therapy application comprises a first application and a second application, wherein the system is constructed and arranged to adapt the second therapy application based on the first therapy application.

86. The system of claim 85 wherein the second therapy application is constructed and arranged to adapt based on the results.

87. The system of claim 85 wherein second therapy application is manually adapted based on the assessment of a speech language pathologist.

88. The system of claim 85 wherein second therapy application is manually adapted based on a patient self-assessment.

89. The system of claim 85 wherein the second therapy application is automatically adapted by the system.

90. The system of claim 89 wherein the system automatic adaptation of the second therapy application is based on at least one of: quantitative analysis of results produced during first therapy application; qualitative analysis of results produced during first therapy application; or indicated clinical pathways based on correlations of application sequencing and improvement types, magnitudes, and change rates.

91. The system of claim 71 wherein the therapy application comprises a series of therapy applications performed prior to the performance of a single therapy application, wherein the single therapy application is adapted based on the multiple therapy applications.

92. The system of claim 91 wherein the single therapy application is adapted based on at least one of: the cumulative results of the multiple therapy applications; the averaged results of the multiple therapy applications; the standard deviation of the results of the multiple therapy applications; or a trending analysis of the results of the multiple therapy applications.

93. The system of claim 1 wherein the system is constructed and arranged to provide the data library to at least one of: the patient; a speech language pathologist; a caregiver; a support group representative; a clinician; a physician; the system manufacturer; a hospital; a health information system; a system component; or a system assembly.

94. The system of claim 1 wherein the data library is at least one of: downloadable; transferable; printable; or recoverable.

95. The system of claim 1 wherein the data library comprises a permission-based access data library.

96. The system of claim 1 wherein the data library comprises a speech-to-text data set of information.

97. The system of claim 96 wherein the speech to text data set comprises a data set customized for a patient diagnosed condition.

98. The system of claim 96 wherein the speech to text data set comprises a data set customized for a patient pre-existing accent.

99. The system of claim 96 wherein the speech to text data set comprises a data set including a limited choice of pre-identified words.

100. The system of claim 1 wherein the data library is constructed and arranged to store data selected from the group consisting of input data; results; patient historic data; external data such as word libraries and medical reference data; and combinations thereof.

101. The system of claim 1 further comprising a configuration algorithm.

102. The system of claim 101 wherein the configuration algorithm is constructed and arranged to configure the system with user-specific modifications.

103. The system of claim 102 wherein the user-specific modifications comprise patient-specific modifications.

104. The system of claim 101 wherein the configuration algorithm is constructed and arranged to allow an operator to set a difficulty level for the system.

105. The system of claim 101 wherein the configuration algorithm modifies one or more system parameters automatically.

106. The system of claim 105 wherein the one or more system parameters are modified based on the results.

107. The system of claim 101 wherein the configuration algorithm modifies one or more system parameters manually.

108. The system of claim 107 wherein the manual modification is performed by at least one of: the patient or a speech and language pathologist.

109. The system of claim 1 further comprising a threshold algorithm.

110. The system of claim 109 wherein the threshold algorithm is constructed and arranged to cause the system to enter an alarm state if one or more parameters fall outside a threshold.

111. The system of claim 109 further comprising a therapy application, wherein the threshold algorithm is constructed and arranged to cause the system to modify the therapy application if one or more parameters fall outside a threshold.

112. The system of claim 109 wherein the threshold algorithm is constructed and arranged to compare a parameter to a threshold wherein the parameter is selected from the group consisting of: lexical parameters such as word length, number of syllables, segmental challenge, phonotactic challenge, abstractness, and age of acquisition; syntactic parameters such as mean length of utterance, phrase structure complexity, and ambiguity metrics; pragmatic parameters such as contextual interpretation support, and salience for particular patient; number or percentage of incorrect answers; number or percentage of correct answers; time taken to perform a task; user input extraneous to completing a task; period of inactivity; time between user input events; hesitation pattern analysis; and combinations thereof.

113. The system of claim 1 further comprising a self-diagnostic assembly.

114. The system of claim 113 wherein the self-diagnostic assembly comprises at least a software algorithm.

115. The system of claim 113 wherein the self-diagnostic assembly comprises at least a hardware assembly.

116. The system of claim 113 wherein the self-diagnostic assembly is constructed and arranged to detect at least one of: power failure; inadequate signal level such as inadequate signal level recorded at the user input assembly; interruption in data gathering; factors affecting human performance such as distraction, fatigue, and aggravation; interruption in data transmission; unexpected system failure; or unexpected termination of a user task.

117. The system of claim 1 further comprising a report generator.

118. The system of claim 117 wherein the report comprises a representation of the results.

119. The system of claim 118 wherein the representation of the results comprises a representation selected from the group consisting of: a graphical representation; a representation of percentages; a representation of comparisons; and combinations thereof.

120. The system of claim 117 wherein the report comprises a comparison of results.

121. The system of claim 120 wherein the comparison of results is selected from the group consisting of: comparison of results from the same patient; comparison of results from the patient and a different patient; comparison of results from the patient to a summary of multiple patient data; and combinations thereof.

122. The system of claim 1 further comprising a non-therapy application.

123. The system of claim 122 wherein the non-therapy application comprises an application selected from the group consisting of: a picture-based electronic communication tool; an audio-based electronic communication tool; a game such as a video game; a news information reader; a telephone internet program; a location-based application using GPS information to provide stimuli for various purposes such as information review, provision of utterance feedback, stimuli to cue functional speech and the like; and combinations thereof.

124. The system of claim 122 wherein the system is constructed and arranged to use the input data to control the non-therapy application.

125. The system of claim 1 further comprising a patient health monitor.

126. The system of claim 125 wherein the patient health monitor is constructed and arranged to detect one or more patient physiologic parameters and/or speech or motor functions indicative of an adverse event based on the input data.

127. The system of claim 126 wherein the adverse event comprises a stroke.

128. The system of claim 1 further comprising a one-click emergency button constructed and arranged to allow a user to contact an emergency handling provider.

129. The system of claim 1 wherein the user input assembly comprises at least one microphone, wherein the system further comprises a mute-detection algorithm constructed and arranged to detect an inadvertent mute condition of the at least one microphone.

130. The system of claim 129 wherein the mute-detection algorithm is further constructed and arranged to contact an emergency handling provider.

131. The system of claim 1 further comprising an automated speech language pathologist function.

132. The system of claim 131 wherein the function is selected from the group consisting of: patient assessment; disease diagnosis; treatment plan creation; delivery of treatment; reporting of treatment delivered; data gathered on patient performance; ongoing reassessment and change to treatment plan; outcome and/or progress prognosis; outcome and/or progress measurement; and combinations thereof.

133. The system of claim 1 further comprising a remote control function.

134. The system of claim 133 wherein the remote control function is constructed and arranged to allow a representative of the manufacturer to perform a function selected from the group consisting of: log in to a system component; control a system component; troubleshoot a system component; train a patient; and combinations thereof.

135. The system of claim 1 further comprising a login function constructed and arranged to allow a user to access the system by entering a username.

136. The system of claim 135 wherein the login function comprises a password algorithm.

137. The system of claim 135 wherein the system is constructed and arranged to allow multiple levels of authorization based on the username.

138. The system of claim 1 further comprising an external input assembly constructed and arranged to receive external data from an external source.

139. The system of claim 138 wherein the input data analyzer is further constructed and arranged to receive the external data, analyze the external data and produce results based on the external data.

140. The system of claim 138 wherein the external input assembly is constructed and arranged to receive the external data via at least one of: a wire; the internet; or a wireless connection such as Bluetooth or cellular service connection.

141. The system of claim 1 further comprising a timeout function constructed and arranged to detect a pre-determined level of user inactivity.

142. The system of claim 141 wherein the system is constructed and arranged to modify a system parameter if a level of user inactivity is detected by the timeout function.

143. The system of claim 141 wherein the system is constructed and arranged to contact an emergency handling provider.

144. The system of claim 1 further comprising other patient data wherein the data analyzer is further constructed and arranged to receive the other patient data and analyze the other patient data.

145. The system of claim 144 wherein the input data analyzer is further constructed and arranged to produce the results based on the other patient data.

146. The system of claim 144 further comprising a therapy application wherein the therapy application is based on the other patient data.

147. The system of claim 144 further comprising a non-therapy application wherein the non-therapy application is based on the other patient data.

148. The system of claim 1 further comprising a family mode.

149. The system of claim 148 further comprising a therapy application wherein the family mode is constructed and arranged to allow a patient family member to participate in the therapy application.

150. The system of claim 1 further comprising a multiple patient mode.

151. The system of claim 150 further comprising a therapy application wherein the multiple patient mode is constructed and arranged to allow multiple patients to participate in the therapy application.

152. The system of claim 1 further comprising a multiple caregiver mode.

153. The system of claim 152 wherein the multiple caregiver mode is constructed and arranged to support multiple users selected from the group consisting of: a therapist such as a speech language therapist; a physical therapist; a psychologist; a general practitioner; a neurologist; and combinations thereof.

154. The system of claim 152 wherein the multiple caregiver mode is constructed and arranged to allow multiple caregivers to access the system at least one of simultaneously or sequentially.

155. The system of claim 1 wherein the system is constructed and arranged to allow transfer of control from a first user to a second user.

156. The system of claim 155 wherein the first user is a patient caregiver and the second user is a patient caregiver, and wherein the system is constructed and arranged to allow transfer of control from the first user to the second user.

157. The system of claim 156 wherein a third user is a patient, and wherein the first user and second user are caregivers of the third user.

158. The system of claim 1 further comprising a billing algorithm.

159. The system of claim 158 wherein the billing algorithm comprises a pay per user algorithm.

160. The system of claim 158 wherein the billing algorithm comprises a pay per time period algorithm.

161. The system of claim 158 wherein the billing algorithm comprises a discount based on at least one of user feedback; extended personal information provided by a user; or user assessments.

162. The system of claim 1 further comprising an email function.

163. The system of claim 1 further comprising a speech to text algorithm.

164. The system of claim 163 wherein the speech to text algorithm comprises an algorithm which is biased by at least one of: accent; disability; slur; stutter; stammer; performance effects of communication disorders such as apraxia of speech, dysarthria, and dysphagia; or organic damage to articulators.

165. The system of claim 1 further comprising a user interface.

166. The system of claim 165 wherein the user interface is constructed and arranged to adapt during use.

167. The system of claim 166 further comprising a diagnostic function wherein the user interface adapts based on the diagnostic function.

168. The system of claim 166 wherein the user interface adapts based on patient performance during use.

169. The system of claim 165 wherein the user interface is manually modified by a user.

170. A method for a patient, comprising:
using the system of claim 1 through 169; and
treating a communication disorder of the patient.

171. The method of claim 170 wherein the method is performed in a specified language.

172. The method of claim 171 wherein the method is performed in English.

173. A method for a patient, comprising:
using the system of claim 1 through 169; and
providing a therapeutic benefit to the patient.

174. A method for a patient, comprising:
using the system of claim 1 through 169; and
providing an orthotic benefit to the patient.

175. A method for a patient, comprising:
using the system of claim 1 through 169; and
providing a prosthetic benefit to the patient.

176. A method for a patient, comprising:
analyzing patient data; and
selecting a therapy based on the analysis.

177. The method of claim 176 wherein the patient data comprises at least recorded speech.

178. The method of claim 177 wherein the recorded speech comprises speech representing at least one of: a sentence; a word; a partial word; a phonetic sound such as a diphone, a triphone or a blend; a phoneme; or a syllable.

179. The method of claim 176 wherein the patient data comprises data selected from the group consisting of: written data; patient movement data such as lip or tongue movement data; patient physiologic data; patient psychological data; patient historic data; and combinations thereof.

180. The method of claim 176 wherein the patient data comprises at least patient written data.

181. The method of claim 180 wherein the patient written data comprises data generated by the patient selected from the group consisting of: a picture; text; written words; icons; symbols; and combinations thereof.

182. The method of claim 176 wherein the patient data comprises at least patient movement data.

183. The method of claim 182 wherein the patient movement data comprises data selected from the group consisting of: data recorded from video camera; data recorded from keypad or keyboard entry; data recorded from mouse movement or clicking; eye movement data; and combinations thereof.

184. The method of claim 176 wherein the patient data comprises at least patient physiologic information.

185. The method of claim 184 wherein the patient physiologic information is selected from the group consisting of: monitored galvanic skin response; respiration; EKG; visual information such as left field cut and right field cut; macular degeneration; lack of visual acuity; limb apraxia; limb paralysis; and combinations thereof.

186. The method of claim 176 wherein the patient data comprises at least patient psychological information.

187. The method of claim 186 wherein the patient psychological information is selected from the group consisting of: Meyer-Briggs personality structure; Enneagram type; disorders such as cognitive disorders and memory disorders; instances of depression; and combinations thereof.

188. The method of claim 176 wherein the patient data comprises at least patient historic data.

189. The method of claim 188 wherein the patient historic data comprises data selected from the group consisting of: patient previous surgery or illness data; family medical history; and combinations thereof.

190. The method of claim 176 wherein the patient data comprises other patient data.

191. The method of claim 176 wherein the patient data comprises external data.

192. The method of claim 176 wherein selecting the therapy can be based on results selected from the group consisting of: diagnostic procedure results; cumulative results of multiple therapy applications; averaged results of multiple therapy applications; standard deviation of results of multiple therapy applications; a trending analysis of results of multiple therapy applications; and combinations thereof.

193. The method of claim 176 wherein selecting the therapy comprises adapting a second therapy application based on a first therapy application.

194. The method of claim 176 wherein the therapy comprises a therapy application and the therapy selection comprises selecting a therapy application level.

195. The method of claim 194 wherein the level comprises at least a first level; a second level more difficult than the first level; and a third level more difficult than the second level.

196. The method of claim 176 wherein the therapy comprises a therapy application and the therapy selection comprises a selection of a therapy sub-application.

197. The method of claim 196 wherein a first therapy sub-application comprises different content than a second therapy sub-application.

198. The method of claim 197 wherein the first therapy sub-application content comprises content selected from the group consisting of: motion picture content; trivia content; sports information content; historic information content; and combinations thereof.

199. The method of claim 196 wherein a first therapy sub-application comprises different functionality than a second therapy sub-application.

200. The method of claim 199 wherein the first therapy functionality comprises a function selected from the group consisting of: phone function such as internet phone function; news retrieval function; word processing function; accounting program function; and combinations thereof.

201. The method of claim 196 wherein a first therapy sub-application comprises different patient provided information than a second therapy sub-application.

202. The method of claim 201 wherein the different patient provided information comprises a difference in at least one of: icons displayed; pictures displayed; text displayed; audio provided; or moving video provided.
